(19) United States
(12) Patent Application Publication
(10) Pub. No.: US 2010/0190242 A1
(43) Pub. Date: Jul. 29, 2010

(54) System for division of a volume of liquid into drops and subsequent drop recollection

(75) Inventors: Andrea Adamo, Cambridge, MA (US); Jennifer Lynn Baltz, Cambridge, MA (US); Luigi Adamo, Cambridge, MA (US)

Correspondence Address: Andrea Adamo, 92 Magazine st-apt 2, Cambridge, MA, 02139, US

(73) Assignees: Dr.Andrea Adamo, Cambridge, MA (US); Dr. Luigi Adamo, Cambridge, MA (US); Mrs. Jennifer Lynn Baltz, Cambridge, MA (US)

(21) Appl. No.: 12/657,555

(22) Filed: Jan. 22, 2010

Related U.S. Application Data
(60) Provisional application No. 61/205,746, filed on Jan. 23, 2009.

(51) Int. Cl. C12M 3/00

(52) U.S. Cl. 435/308.1

(57) ABSTRACT

We describe a system for the division or partition of a volume of fluid into droplets of assigned size and subsequent recollection of the drops.

### CROSS REFERENCE TO RELATED APPLICATIONS

This application claims benefits of U.S. provisional application No. 61/205,746 filed on 1 Jan. 2009, which is hereby incorporated by reference.

### BACKGROUND OF THE INVENTION

Embryoid bodies are aggregates of differentiating pluripotent stem cells and their preparation is a key step for the in vitro production of specific cell types from pluripotent stem cells such as embryonic stem cells and induced pluripotent stem cells (iPS). The gold standard for the preparation of embryoid bodies involves the fractionation of a volume of cell culture medium (a few milliliters to several thousands of milliliters) containing pluripotent cells into hundreds or thousands of hanging drops (each drop with a volume on the order of a few microliters to tens or few hundreds of microliters) where the stem cells self-aggregate and grow, forming said embryoid bodies. At present, said division of large volumes of media is carried out essentially by pipetting, and therefore it involves significant effort and consumption of time.

Stem cell research would benefit from the availability of a method to divide a volume of fluid into hanging drops in a fast and reproducible way, providing a way to save time and money.

### BRIEF SUMMARY OF THE INVENTION

Here we describe a system for the division or partition of a volume of fluid into droplets of assigned size and subsequent recollection of the drops. The system is essentially composed of a set of plates having cavities, with volumes close to the volumes of the drops that are to be made. Each plate is briefly submerged into the fluid to be divided. When the plate is taken out of the fluid, the cavities present in the plate, by means of surface tension, trap part of the fluid inside themselves, thus dividing it into drops. A lid plate and a base plate may be used to protect the plate with the cavities once it is loaded with droplets. The recovery of the drops can be carried out by means of another plate that has structures that enter into each of the holes of the plates and allows removal of the drops. Alternatively the recovery of the drops may be carried out by other methods including, but not limited to, submerging the plates in liquid, flushing the plates with liquid or shaking the plates and causing the droplet to fall.

### BRIEF DESCRIPTION OF THE DRAWINGS

FIG. 1 is a representation of an embodiment of the division plate (100) seen from the top side. A plurality of holes for droplet formation (101) can be recognized

FIG. 2 is a representation of an embodiment of the division plate (100) seen from the bottom side. A plurality of holes for droplet formation (101) can be recognized

FIG. 3 shows a cross section of an embodiment of the division plate (100) seen from the top side. A plurality of holes for droplet formation (101) with conical shape can be recognized

FIG. 4 shows a detailed view of the holes (101) having conical shape

FIG. 5 shows an exploded view of a stack with a cover lid (102), a bottom piece (103) and three division plates (100)

FIG. 6 is a representation of an embodiment of a collection plate (200). A plurality of collection pillars (201) can be recognized

FIG. 7 is a detailed representation of an embodiment of collection pillars (201) having an open, annular cross section

FIG. 8 is a representation of an exploded view of the division plate (100) aligned with a collection plate (200) prior to droplet collection

### DETAILED DESCRIPTION OF THE INVENTION

We describe a system for the division of a volume of fluid into droplets of assigned volume and subsequent recollection of the drops. The division is carried out using at least one “division plate”—fully disclosed further on—that comprises a plurality of holes or cavities for drop trapping. The system can be completed by the use of a specifically designed “recovery plate”—fully disclosed further on—that comprises properly shaped pillars for drop capture.

A division plate is essentially composed of a slab of material (100) having cavities or holes (101) with a volume close to the target volume of the drops. The cavities or holes can be of different shape, for instance, cylindrical or conical. Upon immersion of the slab into a liquid and subsequent removal of the slab from the liquid, drops of fluid are trapped by the holes by means of surface tension. Pinning of the free surface of liquid will occur at the edges of the holes, thus forming and holding drops inside each hole. The wholes can be substituted by indentation or depressions on the surface of the plate.

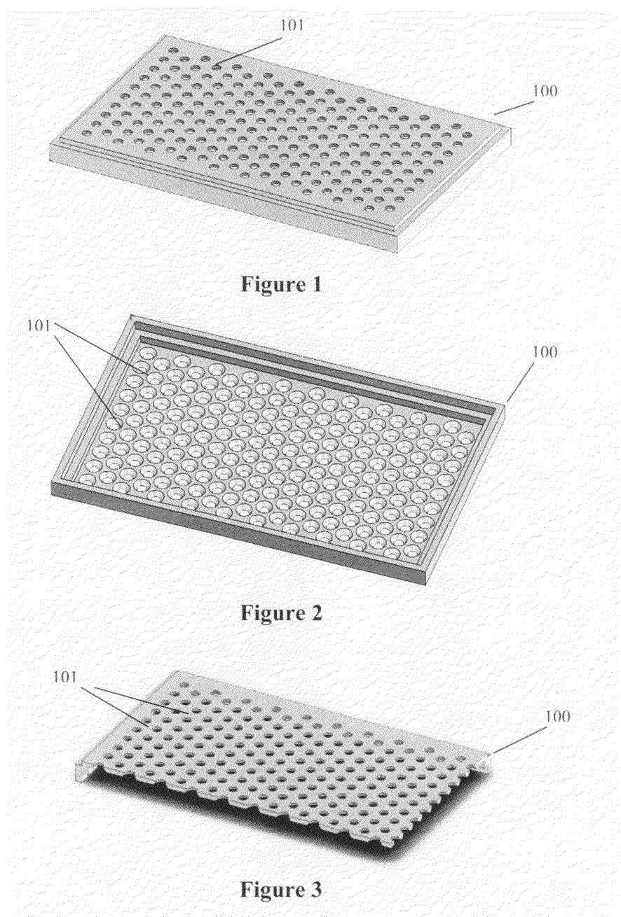

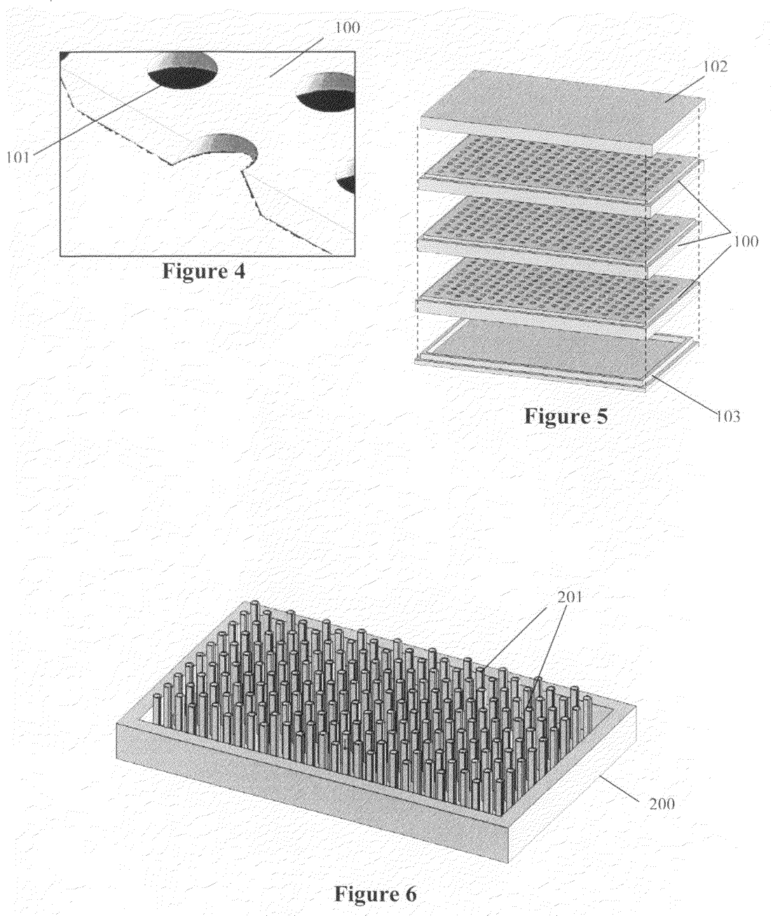

The shape of the cavity, hole or depression (101) does not influence significantly the fractionation process; holes can be cylindrical, conical or any other shape. However, some shapes may be preferred for certain applications. For instance, in the growth of embryoid bodies, hanging drops are used in order to allow the cell to grow in proximity to a liquid boundary instead of a solid one. Therefore, a hole with a conical shape will have an interface on one side with a larger area and that may be beneficial for the growth of embryoid bodies. FIGS. 1, 2 3 show an embodiment of a plate with conical holes.

The plates can have any shape, however, rectangular plates offer the advantage of facilitating plate storage and maximizing the use of space, although other shapes can also be used. For applications in biology, rectangularly shaped plates, that are similar to other sizes of plates already in use in biological research, may be selected to follow standards already used in the marketplace. In this latter case, plates will have an overall height in the millimeter to centimeter range and the other two dimensions would be on the order of a few to several centimeters. Plates may incorporate features that simplify their handling and stacking. Moreover, features that allow or prevent the exchange of gases through gaps, that are found between plates when they are placed in a stack, and the external environment, may be incorporated.

To facilitate the formation of drops, surface properties of the material used for the construction of the plate can be modified in order to change the wettability of the surface. The modification of surface properties can be carried out using chemical or physical methods including, but not limited to, micro and nano patterning of the surface. Surface properties of the entire plate or of just portions of it can be modified to achieve specific behaviors. For instance, the internal surface of the holes can be made hydrophilic to facilitate droplet trapping of aqueous solutions and the rest of the plate can be made hydrophobic to facilitate removal of excess aqueous solution. Alternatively, different materials with different surface properties can be used to assemble a plate.

The plates can be fabricated in any material or combination of materials that is compatible with the selected application. Fabrication will be carried out according to the rules of the art of fabrication with the specific material selected. Some applications, such as applications in biology, may benefit from the use of plastic materials. Some applications may benefit from the use of transparent material, including, but not limited to, polystyrene or polypropylene, for visual inspection of the drops.

The distribution of holes or depressions on the plate can be designed to maximize the density of available areas for drop formation per unit area of the plate. Patterns can be regular or irregular, depending on the overall plate shape and on other specific needs that may be addressed in the plate design.

The application of the described plate for fluid fractionation is not restricted to stem cell research; the concept is rather general and can be used in any field where division of a volume of liquid into droplets is required. Other applications may require plates of different sizes with holes or depressions of different sizes than what is required in stem cell research; this would not result in the loss of the generality of the new invention presented here. For instance, hanging drop formation is also used in the field of protein crystallization, as well as in studies of bacterial motility. Another possible use of the invention is that of droplet formation for high-throughput subdivision of specific substances, per se or before mixing of these aliquots with other substances for downstream applications.

An aspect of novelty of the plate is the exploitation of surface forces to determine droplet formation, thus allowing rapid division of the assigned volume. This concept is novel and rather general therefore other embodiments of the division plate may be conceived. An embodiment of the division plate may have a slab that is not planar, another embodiment may have holes that present corrugation on the edges to improve droplet formation or facilitate droplet removal.

The new concept described here, of exploiting surface forces to determine subdivision of a volume of liquid into droplets, can be used on surfaces that are patterned with areas to be wetted and areas not to be wetted. An embodiment of such a concept may also involve a plate without holes that has a hydrophobic surface with circular hydrophilic spots. Upon immersion of said plate into a liquid volume and subsequent removal, liquid droplets will be trapped by the hydrophilic spots. While such an approach provides division of a volume into droplets, the droplets have a volume that strongly depends on the contact angle of the liquid to the hydrophilic surface; therefore, different liquids will give, on the same plate, droplets of different volume. In contrast, the slab with cavities or holes traps the droplet in the holes, thus providing droplet volumes that depend less on the contact angle of the liquid to the plate.
Another novel aspect of this concept is the fact that the plate contains a number of trapping features thus allowing formation of many droplets at the same time.

The use of the plates for liquid division, involves, but is not limited to, the following operations. A number of plates are immersed in the fluid to be divided, then removed and stacked onto one another (as in FIG. 5). The plates can be put on a custom made base and a custom made cover for the stack can be used in order to protect the drops from contamination or evaporation. The plates can be immersed and emerged one by one or multiple plates can be immersed at the same time.

The liquid division system can be completed by a drop recovery plate (200). A drop recovery plate is essentially composed of a slab of material having pillars (201) coming out of the surface. Said pillars have cross sections that allow them to capture a drop in one of the holes of the fractionation plate once contact between the droplet and the pillar is achieved. In order to achieve contact between the pillars of the recovery plate and the holes of a fractionation plate, pillars are arranged with the same pattern as the holes on the fractionation plate. The capture of the drop is achieved because the size of the pillar is smaller than that of a fractionation hole and capillary forces established between the pillar and the drop capture it on the pillar.

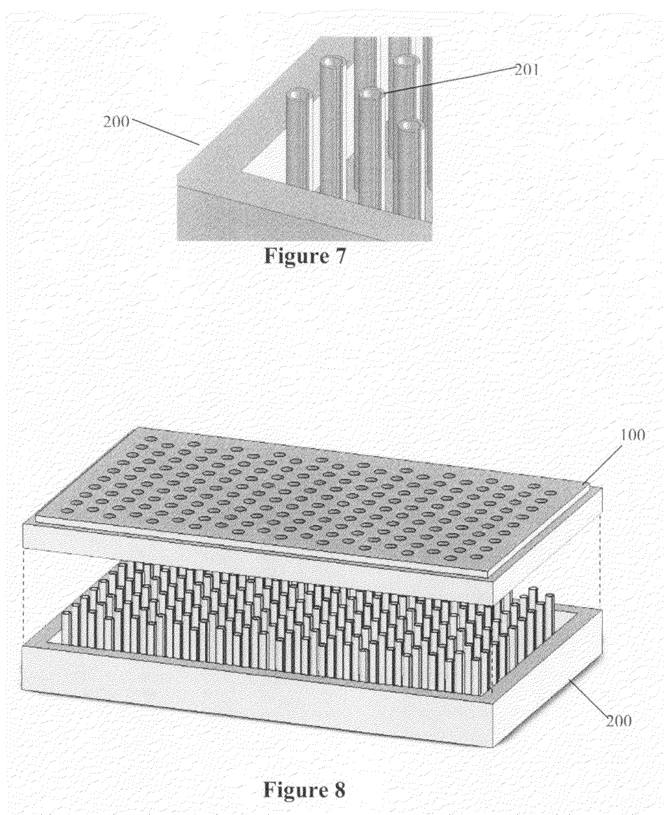

The cross sectional shape of a pillar needs to be such that surface forces will drive the droplet to the pillar. An embodiment of the pillar may include an annular cross section (FIGS. 6, 7) as well as other shapes. A specific pillar shape may be selected for a variety of reasons but, no matter what shape is chosen, the recovery principle, based on the fact that the proper pillar size will determine a preferential behavior of the droplet due to surface forces, is maintained.

A recovery plate can have any shape, however rectangular plates offer the advantage of facilitating storage of the plates and maximizing the use of space, but other shapes can be also be used. For applications in biology, rectangularly shaped plates, that are similar in size to other plates already in use, can be selected to follow standards already used in the marketplace. In this latter case, plates will have a height on the millimeter to centimeter range and the other two dimensions on the order of a few to several centimeters. Plates may incorporate features that simplify their handling and stacking. In order to use a recovery plate in conjunction with a fractionation plate, the same shape and the same distribution of features on the plate could be preferred.

To facilitate the recovery of drops, surface properties of the material used for the construction of the plate can be modified in order to change the wettability of the surface. The modification of surface properties can be carried out using chemical or physical methods, including, but not limited to, micro and nano patterning of the surface. Surface properties of the entire plate or just portions of it can be modified to achieve specific behaviors. For instance, the pillars can have a hydrophilic internal surface to facilitate droplet trapping and a hydrophobic outer surface to facilitate droplet recovery.

The recovery plates can be fabricated in any material or combination of materials that is compatible with the selected application. Fabrication will be carried out according to the rules of the art of the fabrication with the specific material selected. For biological use, plastic materials may be selected; some applications may benefit from transparent material to allow visual inspection of the drops.

The distribution of the pillars on the plate can be designed to maximize the density of available pillars for drop recovery per unit area of the plate. Patterns can be regular or irregular, depending of the overall plate shape and specific needs that may be incorporated into the plate design.

The application of the described plate for droplet recovery is not restricted to the application in stem cell research but the concept is general and can be used in any field where fractionation is required. Different applications may require plates of different sizes having pillars of different size than what required in stem cell research without loss of generality of the new invention presented here.

A recovery plate may contain other features to improve the collection of the recovered drops. In an embodiment of the plate, drops collected from a fractionation plate may slide down to the base of the recovery plate. Additional features as channels or inclined planes can be added to the recovery plate to concentrate the recovered fluid in order to simplify collection of the recovered fluid for further use.

An aspect of novelty of the plate is the exploitation of surface forces to allow capture of a droplet, thus allowing rapid recovery of the liquid. In stem cell research this is a valuable way to recover the droplets in which embryoid bodies have grown. The recovery is important for further use of the embryoid bodies.

The use of recovery plates for recovery of divided liquid volume involves, but is not limited to, the following operations: A division plate containing droplets is brought into contact with a recovery plate either manually or mechanically and, once the droplets have been captured by the recovery plate, the division plate is removed (FIG. 8). If needed, droplets present in other division plates can be removed using the same recovery plate. All the liquid collected by the recovery plate can be removed by the user.

What is claimed is:

1. A device to divide a volume of liquid into smaller aliquots or droplets by means of surface forces.

2. The device of claim 1 where said plate further comprises a plurality of cavities or holes or depressions to trap the liquid.

3. The device of claim 1 and/or 2 where the surface properties of the plate are modified over the entire plate or in parts of it to change its wettability.

4. The device in claim 3 in which the liquid being divided is cell culture medium.

5. The device in claim 3 in which the liquid being divided is a suspension of pluripotent cells, including, but not limited to, embryonic stem cells and induced pluripotent stem cells.

6. The device in claim 3 in which the liquid being divided is a suspension of pluripotent stem cells divided with the purpose of growing embryoid bodies.

7. A device arranged with elements to collect droplets by means of surface forces.

8. The device of claim 7 having pillars as droplet collection elements

9. The device of claim 8 where the pillars have a constant or variable cross section, hollow or not.
